(19) United States
(12) Patent Application Publication
(10) Pub. No.: US 2025/0368585 A1
(43) Pub. Date: Dec. 4, 2025

(54) DEVELOPMENT OF AN EFFICIENT AND PRACTICAL SUSTAINABLE LOWER CARBON AVIATION FUEL (LCAF) FOR IMPROVING AVIATION SUSTAINABILITY

(71) Applicant: HAMAD BIN KHALIFA UNIVERSITY, Doha (QA)

(72) Inventors: Abdulkarem Amhamed, Doha (QA); Rim Romdhane, Doha (QA); Ahmed Sodiq, Doha (QA); Raeesh Muhammad, Doha (QA)

(21) Appl. No.: 19/214,544

(22) Filed: May 21, 2025

Related U.S. Application Data
(60) Provisional application No. 63/652,345, filed on May 28, 2024.

(51) Int. Cl. C07C 1/12; B01D 53/04; B01D 53/26; B01D 53/32; B01J 19/24; C25B 1/042; C25B 15/08; F23C 99/00

(52) U.S. Cl. CPC C07C 1/12; B01D 53/04; B01D 53/261; B01D 53/326; B01J 19/2465; C25B 1/042; C25B 15/081; F23C 99/00; B01D 2257/504; B01D 2257/80; B01D 2258/0283; B01J 2219/00045; F23C 2900/99011

(57) ABSTRACT

A carbon closed-loop system and process are provided. The carbon closed-loop system and process can be utilized in an industrial operation for producing, for example, a Lower Carbon Aviation Fuel (LCAF). The LCAF is produced by decarbonizing, for example, industrial furnaces and boilers, such as fired heaters, through the carbon closed-loop system and process which integrates renewable energy-driven H₂ generation, CO₂ capture, and methanation technologies to substantially reduce the carbon footprint of the industrial operation.

### CROSS REFERENCE TO RELATED APPLICATIONS

The present application claims priority to U.S. Provisional Patent Application No. 63/652,345, filed on May 28, 2024, the entire contents of which are incorporated herein by reference.

### BACKGROUND

A problem being addressed, for example, is the significant impacts on global greenhouse gas (GHG) emissions, alongside the challenges faced in scaling up the production, for example, in the transportation industry. Sustainable Aviation Fuels (SAF) help meet growing demands, such as those related to travel (e.g., air, land, and water travel), without compromising environmental standards. For example, reducing greenhouse gas emissions and decarbonizing industrial processes is a pressing imperative driven by the urgent need to mitigate climate change, promote environmental sustainability, comply with regulations, seize economic opportunities, enhance energy security, and fulfill our societal responsibility. The aviation industry's significant contribution to global GHG emissions, coupled with the heavy reliance of energy-intensive industries on fossil fuels, necessitates concerted efforts to develop sustainable alternatives.

Addressing this problem, for example, aligns with the global effort to minimize environmental impact, preserve natural resources, comply with emissions targets, and transition towards a low-carbon economy. It presents economic opportunities, enhances energy security through diversification, and aligns with the ethical imperative to protect our planet and ensure a sustainable future.

### SUMMARY

According to a non-limiting aspect of the present disclosure, an exemplary embodiment of a method of using a carbon closed-loop system in an industrial operation is provided. In an embodiment, the method includes capturing, in a direct flue gas electrolysis (DFGE) unit, a water vapor from a flue gas from a furnace used in the industrial operation to produce green hydrogen and oxygen. Further, the method includes capturing, in a carbon removal unit, carbon dioxide from the flue gas. The method further includes feeding the carbon dioxide and the green hydrogen to a hydrogenation unit, wherein the hydrogenation unit includes a methanation process to produce green methane from the carbon dioxide and the green hydrogen. Further, the method includes utilizing the green methane and the oxygen in the furnace to enhance combustion efficiency and reduce greenhouse gas emissions.

In a second aspect of the present disclosure, which may be combined with any other aspect listed herein unless specified otherwise, the carbon closed-loop system is integrated with the industrial operation for manufacturing a Lower Carbon Aviation Fuel (LCAF).

In a third aspect of the present disclosure, which may be combined with any other aspect listed herein unless specified otherwise, a hygroscopic material (such as concentrated sulfuric acid (H₂SO₄), potassium hydroxide (KOH), sodium hydroxide (NaOH), ammonium nitrate (NH₄NO₃), potassium phosphate (K₃PO₄), potassium ethanoate (CH₃COOK), chloride of calcium, magnesium, zinc, iron, and potassium carbonate (K₂CO₃)) is utilized in the DFGE unit to capture the water vapor from the flue gas.

In a fourth aspect of the present disclosure, which may be combined with any other aspect listed herein unless specified otherwise, the water vapor is electrolyzed in the DFGE unit to produce the green hydrogen.

In a fifth aspect of the present disclosure, which may be combined with any other aspect listed herein unless specified otherwise, carbon dioxide is captured in the carbon removal unit that includes a sorbent material (mesoporous silica functionalized with amines or ionic liquids, high surface area porous polymers, metal-organic frameworks (MOFs), heteroatom-functionalized activated carbons, zeolites, alkali metal oxides, mixed metal oxides, and functionalized composites of the aforementioned porous materials with alkali metals or metal oxides) through which the flue gas is fed from the DFGE unit.

In a sixth aspect of the present disclosure, which may be combined with any other aspect listed herein unless specified otherwise, the industrial operation is in a water-scarce region.

In a seventh aspect of the present disclosure, which may be combined with any other aspect listed herein unless specified otherwise, the sorbent material has a high CO₂ adsorption capacity and selectivity.

In an eighth aspect of the present disclosure, which may be combined with any other aspect listed herein unless specified otherwise, the industrial operation includes a distillation tower.

In a nineth aspect of the present disclosure, which may be combined with any other aspect listed herein unless specified otherwise, the carbon closed-loop system includes renewable energy-driven H₂ generation.

According to another non-limiting aspect of the present disclosure, an exemplary embodiment of a carbon closed-loop system for an industrial operation including a furnace that generates a flue gas is provided. In an embodiment, the carbon closed-loop system comprises a direct flue gas electrolysis (DFGE) unit configured to capture a water vapor from the flue gas from the furnace to produce green hydrogen and oxygen, a carbon removal unit configured to receive the flue gas from the DFGE unit to capture carbon dioxide from the flue gas, and a hydrogenation unit configured to receive the carbon dioxide and the green hydrogen, wherein the hydrogenation unit is operable to produce green methane, via methanation, from the carbon dioxide and the green hydrogen, thereby utilizing the green methane and the oxygen in the furnace to enhance combustion efficiency and reduce greenhouse gas emissions.

In an eleventh aspect of the present disclosure, which may be combined with any other aspect listed herein unless specified otherwise, the carbon closed-loop system is integrated with the industrial operation for manufacturing a Lower Carbon Aviation Fuel (LCAF).

In a twelfth aspect of the present disclosure, which may be combined with any other aspect listed herein unless specified otherwise, a hygroscopic material is utilized in the DFGE unit, and wherein the hygroscopic material is configured to capture the water vapor from the flue gas.

In a thirteenth aspect of the present disclosure, which may be combined with any other aspect listed herein unless specified otherwise, the DFGE unit is configured to electrolyze the water vapor to produce the green hydrogen.

In a fourteenth aspect of the present disclosure, which may be combined with any other aspect listed herein unless specified otherwise, the carbon removal unit is configured to capture carbon dioxide, and wherein the carbon removal unit includes a sorbent material.

In a fifteenth aspect of the present disclosure, which may be combined with any other aspect listed herein unless specified otherwise, the industrial operation is located in a water-scarce region.

In a sixteenth aspect of the present disclosure, which may be combined with any other aspect listed herein unless specified otherwise, the sorbent material has a high CO₂ adsorption capacity and selectivity.

In a seventeenth aspect of the present disclosure, which may be combined with any other aspect listed herein unless specified otherwise, the industrial operation includes a distillation tower.

In an eighteenth aspect of the present disclosure, which may be combined with any other aspect listed herein unless specified otherwise, the carbon closed-loop system includes a renewable energy-driven H₂ generation source.

Additional features and advantages are described herein, and will be apparent from the following Detailed Description.

### BRIEF DESCRIPTION OF THE FIGURES

The specification makes reference to the following figures, in which use of like reference numerals in different figures is intended to illustrate like or analogous components.

FIG. 1 illustrates an example embodiment of a carbon closed-loop system and process utilized with an industrial operation.

FIGS. 2A and 2B illustrate an Aspen Plus V14 simulation (FIG. 2A) that was conducted to investigate feasibility and potential benefits of an example carbon closed-loop system and process (FIG. 2B).

Various implementations described herein may include additional systems, methods, features, and advantages, which cannot necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

### DETAILED DESCRIPTION

The present disclosure generally relates to a carbon closed-loop system and process. The carbon closed-loop system and process can be utilized in an industrial operation for producing, for example, a Lower Carbon Aviation Fuel (LCAF). In an embodiment, the LCAF is produced by decarbonizing, for example, industrial furnaces and boilers, such as fired heaters, through the carbon closed-loop system and process, which integrates renewable energy-driven H₂ generation, CO₂ capture, and methanation technologies to substantially reduce the carbon footprint of industrial operations. The carbon closed-loop technology will be described below in further detail including in reference to the figures according to various embodiments.

### Direct Flue Gas Electrolysis (DFGE) for Green Hydrogen Production

The carbon closed-loop technology begins with capturing water vapor from flue gas using a hygroscopic material. The hygroscopic material is selected based on efficiency in capturing water vapor from the flue gas stream. The captured water vapor is then directed to a suitable electrode material and a compatible membrane for water electrolysis and hydrogen separation. The captured water vapor is electrolyzed to produce green hydrogen, which is then separated from the electrolysis cell using the membrane. The DFGE system is designed and optimized to maximize green hydrogen production while minimizing energy consumption.

### CO₂ Capture Using Novel Sorbent Materials

The carbon closed-loop technology further includes a carbon removal unit that includes a novel sorbent material for efficient CO₂ capture in dry conditions. The sorbent material is designed and screened to have high CO₂ adsorption capacity and selectivity. The flue gas stream from DFGE is directed through a CO₂ capture unit containing the sorbent material, where CO₂ is selectively adsorbed and separated from other gases in the flue gas stream.

### Low-Temperature Methanation for Green Methane Production

The captured CO₂ and produced green hydrogen are then used in a low-temperature methanation process to produce green methane. The methanation process involves a high-performance catalyst designed to facilitate the reaction between CO₂ and hydrogen at low temperatures. The green methane produced in this process can be utilized in the furnace, replacing conventional fossil fuels and reducing greenhouse gas emissions.

### Oxygen Production and Utilization

During the DFGE process, oxygen is also produced as a byproduct. This oxygen can be utilized in the furnace along with the green methane, improving combustion efficiency and further reducing greenhouse gas emissions.

The carbon closed-loop technology has several key aspects according to an embodiment. First, the carbon closed-loop technology provides an integrated system design and optimization for efficiency and energy consumption. Next, the carbon closed-loop technology provides advancements in DFGE technology, particularly related to water vapor capture and catalyst optimization. Further, the carbon closed-loop technology utilizes a novel sorbent material for efficient CO₂ capture in dry flue gas conditions. Moreover, the carbon closed-loop technology provides integration strategies for adapting the system/process to handle conventional flue gas and produce green methane to align with the industrial operation requirements, such as LCAF requirements.

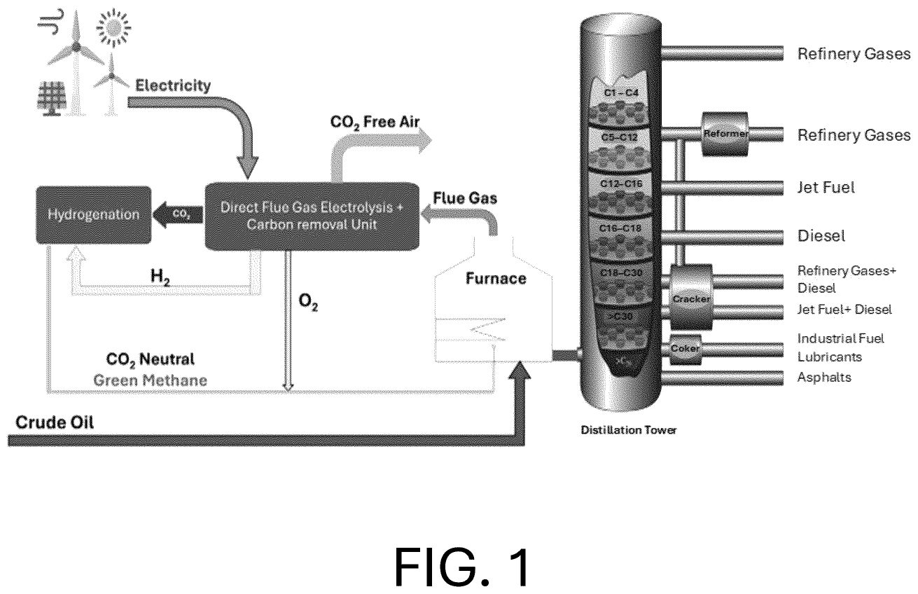

FIG. 1 illustrates an example embodiment of a carbon closed-loop system and process. FIG. 1 illustrates a carbon closed-loop system and process that is integrated with an industrial operation, namely, an LCAF industrial operation. In FIG. 1, a distillation tower is connected to a furnace to which crude oil is sent to heat and operate the distillation tower to produce a number different products including jet fuel. The carbon closed-loop technology is integrated with the industrial operation via the furnace which generates and emits flue gas to the carbon closed-loop technology that includes a DFGE unit, a carbon removal unit and a hydrogenation unit. The carbon closed-loop technology produces green methane from flue gas using DFGE, CO₂ capture, and methanation processes. Water vapor is captured from flue gas using a hygroscopic material, followed by electrolysis to generate green hydrogen. A novel sorbent material is utilized for efficient CO₂ capture in dry conditions. The captured CO₂ and produced hydrogen are then used in a low-temperature methanation process in the hydrogenation unit to produce green methane, which can be utilized in the furnace along with oxygen produced during electrolysis in the DFGE unit.

In the illustrated carbon closed-loop system and process, the required energy is produced from renewable energy-driven H₂ generation. The carbon closed-loop system and process can be used to heat the furnace, which can provide the necessary heat energy to operate an industrial operation including a variety of machines and/or processes, such as a distillation tower, a reformer, a cracker, a coker, suitable other industrial operation units and suitable combinations thereof. In an illustrative embodiment, the closed-loop system and process can be used to generate heat, which maintains a distillation tower that produces refinery gases, gasoline, jet fuel, diesel, refiner gases, industrial fuel lubricants, and asphalt from crude oil. The distillation tower can also include various components to increase the purity of the products such as packing, reboilers and condensers, and/or trays. As an illustrative example, a furnace or another heating component can provide energy in the form of heat to the distillation column, which allows for separation of components/products based on varying boiling points as shown in FIG. 1.

The carbon closed-loop system and process begin with capturing flue gas, which is produced as a byproduct from a heating unit (e.g., furnace) used. First, the captured flue gas will enter the DFGE unit, which captures water vapor from flue gas using one or more hygroscopic materials. The hygroscopic material is selected based on efficiency in capturing water vapor from the flue gas stream. The captured water vapor is then directed to a suitable electrode material and a compatible membrane for water electrolysis and hydrogen separation. The captured water vapor is electrolyzed to produce green hydrogen, which is then separated from the electrolysis cell using a suitable membrane. The DFGE system of the carbon closed-loop system and process is designed and optimized to maximize green hydrogen production while minimizing energy consumption.

Additionally, the DFGE system of the carbon closed-loop system and process produces captured CO₂ via a carbon removal unit as illustrated in FIG. 1. The CO₂ is captured using a novel sorbent material. For example, the carbon closed-loop system and process utilizes a novel sorbent material for efficient CO₂ capture in dry conditions. The sorbent material is designed and screened to have high CO₂ adsorption capacity and selectivity. The flue gas stream from DFGE is directed through a CO₂ capture unit (carbon removal unit) containing the sorbent material, where CO₂ is selectively adsorbed and separated from other gases in the flue gas stream.

Further, the captured CO₂ and produced green hydrogen are directed in a hydrogenation step of the carbon closed-loop system and process. In the hydrogenation step, the captured CO₂ and produced green hydrogen are used in a low-temperature methanation process to produce green methane. The methanation process involves a high-performance catalyst designed to facilitate the reaction between CO₂ and hydrogen at low temperatures. The green methane produced in this process can be utilized as an energy (heating) source for the furnace, replacing conventional fossil fuels and reducing greenhouse gas emissions. During the DFGE process, oxygen is also produced as a byproduct. This oxygen can be utilized in the furnace along with the green methane, improving combustion efficiency and further reducing greenhouse gas emissions, as further illustrated in FIG. 1.

The green methane and oxygen byproduct can then be used as an energy source for a heating element, such as a heating element in the furnace. The heating element can be used in a variety of applications such as providing energy to chemical processes for solubility enhancements, as a reactant, or controlling the temperature of certain equipment. In an illustrative example, a distillation tower is heated with the furnace, which separates the components in the tower based on varying boiling points. For example, the component with the highest boiling point (requires a greater temperature to boil) will leave the distillation column closer to the bottom and the component with the highest boiling (requires a smaller temperature to boil) will leave the distillation column closer to the top. The distillation tower can also include various components to increase the purity of the purity of the products such as packing, reboilers and condensers, and/or trays.

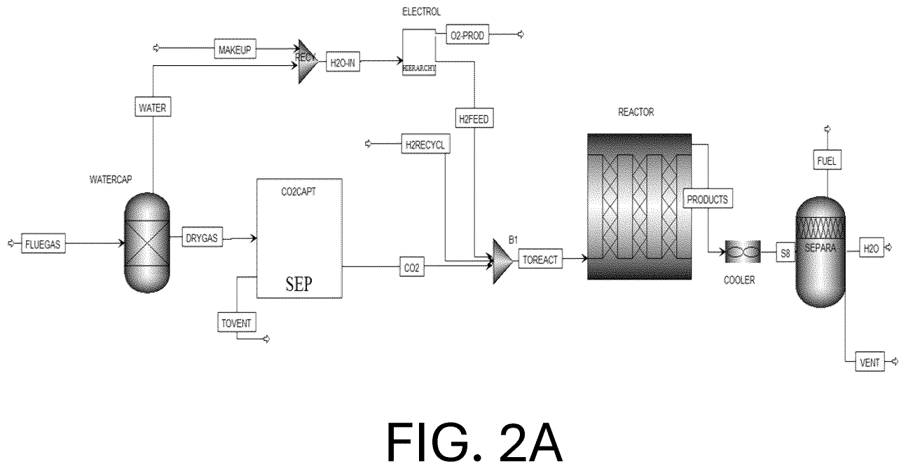

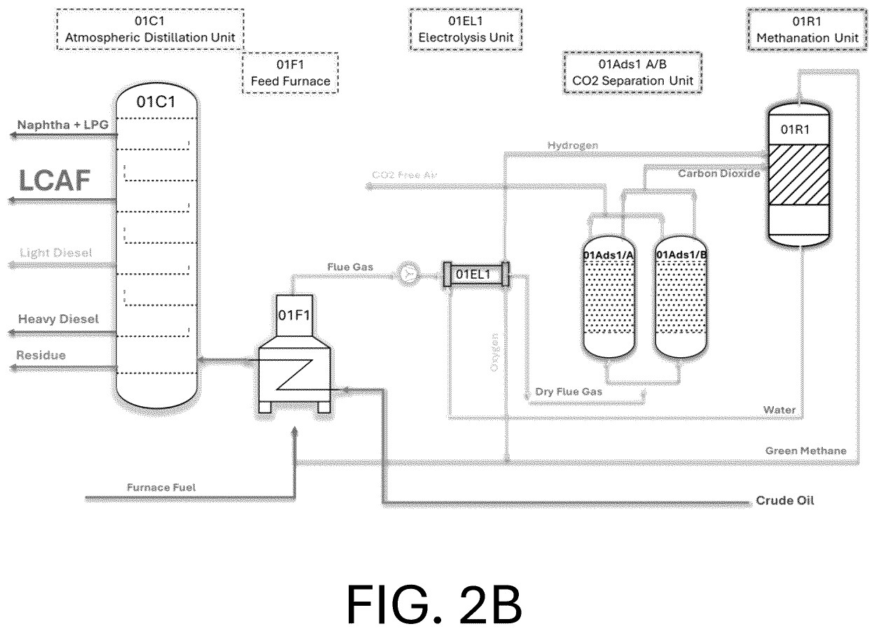

FIGS. 2A and 2B illustrate an Aspen Plus V14 simulation (FIG. 2A) that was conducted to investigate feasibility and potential benefits of an example carbon closed-loop system and process (FIG. 2B). The Aspen Plus V14 simulation provided modeling of the optimization of the various process steps, including electrolysis, CO₂ capture, and methanation, as well as to evaluate the overall process performance and identify potential synergies between the different system/process features of the carbon closed-loop technology. The carbon closed-loop system and process simulation focused on several key aspects. From this simulation, it was determined that the carbon closed-loop system and process can achieve substantial cost reductions and efficiency improvements. The key findings from the simulations are included in the following description.

First, water from flue gas and methanation can be used directly in an electrolyzer for hydrogen production, which eliminates the need for costly water treatment and enables the carbon closed-loop system and process to work efficiently and effectively even in water-scarce regions. Water-scarce regions are regions where the availability of freshwater resources is lower than the demand, which could lead to a shortage of freshwater resources. Further, the exothermic nature of the Sabatier reaction releases substantial energy, which can be used for CO₂ sorbent regeneration, enhancing overall efficiency, and reducing operational costs.

Despite the current efficiency limitations of the Power-to-Methane-to-Power process, which typically range from 30% to 38%, the carbon closed-loop technology can enable the replacement of at least 60% of the fuel consumed in the same furnace with green fuel generated from its own waste gases. This substantial replacement potential underscores the effectiveness and sustainability of our approach, as it allows for a significant reduction in the reliance on conventional fossil fuels.

Moreover, using O₂ produced from electrolysis in the combustion improves combustion efficiency, reduces fuel consumption, lowers energy losses, decreases emissions of pollutants like nitrogen oxides (NOx), reducing operating costs and allows for better process control and flexibility.

During the furnace startup process, the carbon closed-loop system and process, for example, can further include preventing contaminants from entering the electrolysis unit. This is achieved by initially bypassing the electrolysis unit, allowing flue gas to directly undergo CO₂ sorption. Treated water from the refinery utilities unit can be used for the electrolyzer until green methane is produced. The green methane can then be utilized in the furnace with the produced O₂, effectively reducing electrolysis contaminants, such as, SO_{X} and NO_{X} in the flue gas. After this phase, the electrolysis process can be reintroduced, using water harvested from the flue gas, ensuring a sustainable and efficient startup process.

As discussed above, solving the problem of reducing greenhouse gas emissions from an industrial process (e.g., aviation fuel production) and decarbonizing industrial processes is a pressing need, which is driven by the urgent need to mitigate climate change, promote environmental sustainability, comply with regulations, seize economic opportunities, enhance energy security, and fulfill our societal responsibility. The aviation industry's significant contribution to global GHG emissions, coupled with the heavy reliance of energy-intensive industries on fossil fuels, necessitates concerted efforts to develop sustainable alternatives.

To address this problem, the carbon closed-loop system and process integrates a number of technologies. For example, the carbon closed-loop system and process integrates three pivotal technologies according to an embodiment. First, renewable energy sources, such as solar or wind power, are used to drive the electrolysis of captured water from flue gas, producing green hydrogen with a significantly lower carbon footprint compared to conventional methods of hydrogen production. Next, the illustrated system incorporates efficient carbon capture technologies to capture CO₂ emissions from industrial processes, particularly from furnaces and boilers, which are major contributors to refinery emissions. Further, the captured CO₂ will be combined with the green hydrogen through the Sabatier reaction, a methanation process, to produce synthetic methane (green methane). The green methane can serve as a drop-in fuel, replacing conventional fossil fuels in existing infrastructure.

By integrating such technologies, the illustrated system aims to create a closed-loop cycle where CO₂ emissions from industrial processes are captured and converted into green methane fuel, which can then be used to power the same processes. This significantly reduces the carbon footprint. Furthermore, the carbon closed-loop technology leverages the oxygen byproduct from the electrolysis process to enhance combustion efficiency in industrial furnaces and boilers through oxygen-enriched combustion. This will lead to reduced fuel consumption and lower emissions, further contributing to decarbonization efforts. This approach not only addresses the emissions from the aviation sector by providing a pathway for the LCAF but also targets the decarbonization of energy-intensive processes, particularly in hard-to-abate industries, which are significant contributors to global greenhouse gas emissions.

The illustrative process, in an embodiment, provides the following aspects: innovative technologies, using water from the process, process integration, targeting the decarbonization of furnaces and boilers, and providing a pathway for LCAF, which can complement SAF (Sustainable Aviation Fuel) according to an embodiment.

For example, the illustrated process integrates three innovative technologies: renewable energy-driven hydrogen generation, carbon capture, and methanation according to an embodiment. These technologies are integrated into a carbon closed-loop system tailored for industrial applications. Further, by capturing and utilizing water vapor from industrial flue gases and from the methanation reactor, the illustrated process eliminates the need for costly treated water, reducing operational expenses and enhancing resource efficiency. The water harvesting technique is also a unique feature of the process.

Moreover, the illustrated process is designed for seamless integration with existing industrial processes, which enables the efficient utilization of energy and byproducts. For instance, the oxygen generated during electrolysis is used for oxygen-enriched combustion, improving furnace and boiler efficiency, the produced water and the energy released during the methanation reaction are reintegrated to the process to enhance the process overall efficiency.

The illustrated process is also targeted at the decarbonization of furnaces and boilers, which are responsible for a significant portion of refinery emissions. By capturing CO₂ from these sources and converting it into green fuel, our approach is directed to address a challenging and often overlooked aspect of refinery (and other fossil fuel based industries) emissions.

Finally, the relevancy to the aviation sector is important. By producing green methane, the illustrated system provides a pathway for LCAF, which can complement SAF in reducing emissions from the aviation sector while meeting the stringent CORSIA sustainability criteria.

The subject matter of embodiments is described herein with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1: A method of using a carbon closed-loop system in an industrial operation, the method comprising:
capturing, in a direct flue gas electrolysis (DFGE) unit, a water vapor from a flue gas from a furnace used in the industrial operation to produce green hydrogen and oxygen;
capturing, in a carbon removal unit, carbon dioxide from the flue gas;
feeding the carbon dioxide and the green hydrogen to a hydrogenation unit, wherein the hydrogenation unit includes a methanation process to produce green methane from the carbon dioxide and the green hydrogen; and
utilizing the green methane and the oxygen in the furnace to enhance combustion efficiency and reduce greenhouse gas emissions.

2: The method of claim 1, wherein the carbon closed-loop system is integrated with the industrial operation for manufacturing a Lower Carbon Aviation Fuel (LCAF).

3: The method of claim 1, wherein a hygroscopic material is utilized in the DFGE unit to capture the water vapor from the flue gas.

4: The method of claim 1, wherein the water vapor is electrolyzed in the DFGE unit to produce the green hydrogen.

5: The method of claim 1, wherein the carbon dioxide is captured in the carbon removal unit that includes a sorbent material thru which the flue gas is fed from the DFGE unit.

6: The method of claim 5, wherein the sorbent material has a high CO₂ adsorption capacity and selectivity.

7: The method of claim 1, wherein the industrial operation is in a water-scarce region.

8: The method of claim 1, wherein the industrial operation includes a distillation tower.

9: The method of claim 1, wherein the carbon closed-loop system includes renewable energy-driven H₂ generation.

10: A carbon closed-loop system for an industrial operation including a furnace that generates a flue gas, the carbon closed-loop system comprising:
a direct flue gas electrolysis (DFGE) unit configured to capture a water vapor from the flue gas from the furnace to produce green hydrogen and oxygen;
a carbon removal unit configured to receive the flue gas from the DFGE unit to capture carbon dioxide from the flue gas; and
a hydrogenation unit configured to receive the carbon dioxide and the green hydrogen, and wherein the hydrogenation unit is operable to produce green methane, via methanation, from the carbon dioxide and the green hydrogen, thereby utilizing the green methane and the oxygen in the furnace to enhance combustion efficiency and reduce greenhouse gas emissions.

11: The carbon closed-loop system of claim 10, wherein the carbon closed-loop system is integrated with the industrial operation for manufacturing a Lower Carbon Aviation Fuel (LCAF).

12: The carbon closed-loop system of claim 10, wherein a hygroscopic material is utilized in the DFGE unit, and wherein the hygroscopic material is configured to capture the water vapor from the flue gas.

13: The carbon closed-loop system of claim 10, wherein the DFGE unit is configured to electrolyze the water vapor to produce the green hydrogen.

14: The carbon closed-loop system of claim 10, wherein the carbon removal unit is configured to capture carbon dioxide, and wherein the carbon removal unit includes a sorbent material.

15: The carbon closed-loop system of claim 14, wherein the sorbent material has a high CO₂ adsorption capacity and selectivity.

16: The carbon closed-loop system of claim 10, wherein the industrial operation is located in a water-scarce region.

17: The carbon closed-loop system of claim 10, wherein the industrial operation includes a distillation tower.

18: The carbon closed-loop system of claim 10, wherein the carbon closed-loop system includes a renewable energy-driven H₂ generation source.
